(19) United States
(12) Patent Application Publication
(10) Pub. No.: US 2021/0326811 A1
(43) Pub. Date: Oct. 21, 2021

(54) Time Clock with Thermal Reader Integral Therewith

(71) Applicant: Ascentis Corporation, Eden Prairie, MN (US)

(72) Inventors: Zeus Chen, Rancho Cucamonga, CA (US); Ting Chuang, Rancho Cucamonga, CA (US); Yevgeniy Goyfman, Eden Prairie, MN (US)

(73) Assignee: Ascentis Corporation, Eden Prairie, MN (US)

(21) Appl. No.: 17/083,661

(22) Filed: Oct. 29, 2020

Related U.S. Application Data
(60) Provisional application No. 63/012,508, filed on Apr. 20, 2020.

(51) Int. Cl. G06Q 10/10; A61B 5/01; A61B 5/00; G07C 9/00

(52) U.S. Cl. CPC G06Q 10/1091; A61B 5/015; A61B 2562/0271; G07C 9/00182; A61B 5/6887

(57) ABSTRACT

A time-clock system, including a thermal reader, configured to handle employee clock-in and clock-out events, acquire a body temperature reading of an employee before that employee clocks in for work, prevent clocking in and entry into a workplace if a temperature threshold is exceeded, and track employee contacts through the work period. The time-clock system is designed to improve the health and safety of employees and reduce the risk spreading an infection, including COVID-19, to other employees or adversely affect the employer's workflow and productivity. The system may also be used to perform contact tracing to minimize the extent of outbreaks of infectious diseases, including COVID-19, in the workplace.

### CROSS-REFERENCE TO RELATED APPLICATION(S)

This application claims the benefit of U.S. Provisional Patent Application Ser. No. 63/012,508 entitled “TIME CLOCK WITH THERMAL READER INTEGRAL THEREWITH,” filed Apr. 20, 2020, which application is hereby incorporated by this reference in its entirety.

### TECHNICAL FIELD

The invention generally pertains to a time-clock for recording employee clock-in and clock-out times. In particular, the invention pertains to a time-clock with a thermal reader for acquiring an employee temperature during a clock-in or clock-out event.

### BACKGROUND

Numerous employees use time-clocks to clock-in and clock-out for purposes of documenting hours worked. In the era of COVID-19, however, these time-clocks do nothing to assess the risk of employees coming to work with an infectious disease, or assess the risk of exposure to other employees. There is, therefore, a need for a technique and device to control the risk of introducing and spreading infectious diseases, including COVID-19, in the workplace.

### SUMMARY

The present invention is directed to a time-clock system configured to handle employee clock-in and clock-out events, perform health checks, and track contacts through the work period. The tracking function can then be used to perform contact tracing to minimize the extent of outbreaks of infectious diseases in the workplace.

In the preferred embodiment, the present invention is a system including both a time-clock and thermal reader. The thermal reader is configured to acquire a body temperature reading of an employee before that employee clocks in for work. The time-clock then checks the temperature reading and prevents the employee from clocking in to work if the employee's temperature is above a predetermined threshold, for example. Moreover, the time-clock also checks the temperature reading of employees at time of clock-out, thereby narrowing down the scope of deep-sanitation efforts and contact-tracing in cases where an employee is infected with infectious disease like COVID-19.

These and other features, aspects, and advantages of the present invention will become better understood with reference to the following description and appended claims.

### BRIEF DESCRIPTION OF DRAWINGS

FIG. 1 is functional block diagram of the thermal-reader time-clock (TRTC) system;

FIG. 2 is diagrammatic illustration of a thermal reader;

FIG. 3 is a front side view of the thermal reader mounted to the time-clock;

FIG. 4 is a back side view of the thermal reader mounted to the time-clock;

FIG. 5 is an exploded view of the thermal reader mounted to the time-clock;

FIG. 6 is a procedure for initialization of a thermal reader after installation on a time-clock;

FIG. 7 is procedure for acquiring an employee's consent to a temperature measurement using a time-clock;

FIG. 8 is a procedure for acquiring an employee's consent to a temperature measurement using a web interface;

FIG. 9 is a procedure for providing identifying information to a time-clock;

FIG. 10 shows the TRTC system acquiring an image and body temperature reading of an employee;

FIG. 11 is an image composed of a plurality of cells, each cell including a body temperature reading for that section of an image;

FIG. 12 is a procedure to determine if an employee temperature reading should be taken;

FIG. 13 is a procedure for ensuring employee consent to a temperature reading;

FIG. 14 is a procedure to be implemented when an employee consents to a temperature reading;

FIG. 15 is procedure to be implemented when a temperature reading results in “reading failed”;

FIG. 16 is procedure to be implemented when a temperature reading exceeds a temperature threshold;

FIG. 17 is procedure to be implemented when a control reading does not exceed the established temperature threshold defined for that employee; and

FIGS. 18A-18I is a sequence of dialogue boxes through which an employer defines various actions to be taken based, in part, on an employee's body temperature.

### DETAILED DESCRIPTION OF THE INVENTION

The detailed description set forth below in connection with the appended drawings is intended as a description of presently-preferred embodiments of the invention and is not intended to represent the only forms in which the present invention may be constructed or utilized. The description sets forth the functions and the sequence of steps for constructing and operating the invention in connection with the illustrated embodiments. It is to be understood, however, that the same or equivalent functions and sequences may be accomplished by different embodiments that are also intended to be encompassed within the spirit and scope of the invention.

The present invention in the preferred embodiment is time clock system that enables employers to implement a customizable set of workplace health and safety measures that reduce the risk of introduction and/or spread of infectious diseases at a work facility and empower the employers to implement containment and mitigation in situations in which infected individuals are identified after they have gained access to the workplace. In the preferred embodiment, the present invention features a time-clock with thermal reader (herein referred to as the thermal-reader time-clock (TRTC) system) for reading the body temperature of an employee when the employee is clocking-in for work. Once the temperature is read, an employee database or other memory device is queried to identify a predetermined response base on the temperature reading. If the temperature is within normal levels, the TRTC system enables the employee to clock in for work. If the temperature reading indicates that the employee could have a fever, for example, the TRTC system may prevent the employee from clocking in and take any of a plurality of optional actions. In this manner, the TRTC system can improve the health and safety of employees and reduce the risk spreading an infection to other employees or adversely affect the employer's workflow.

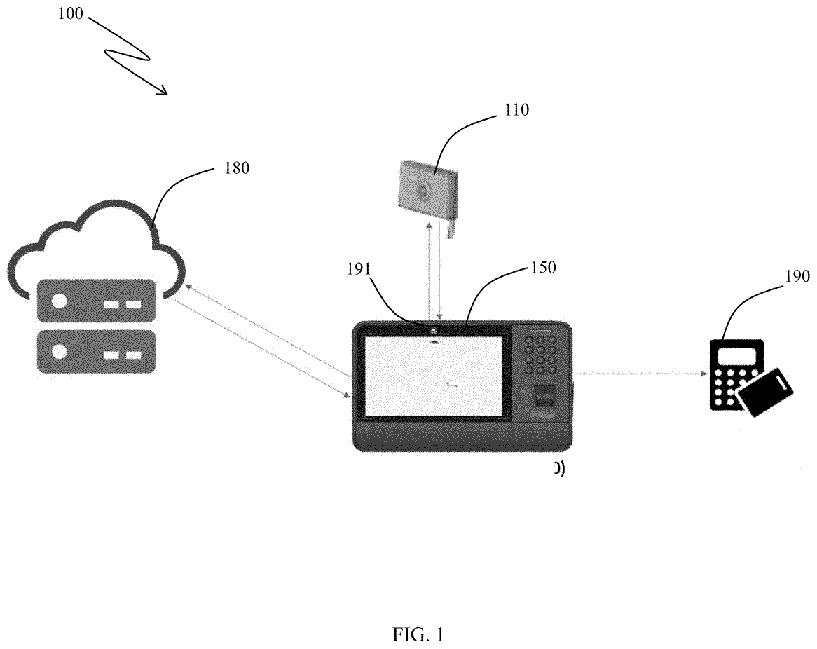

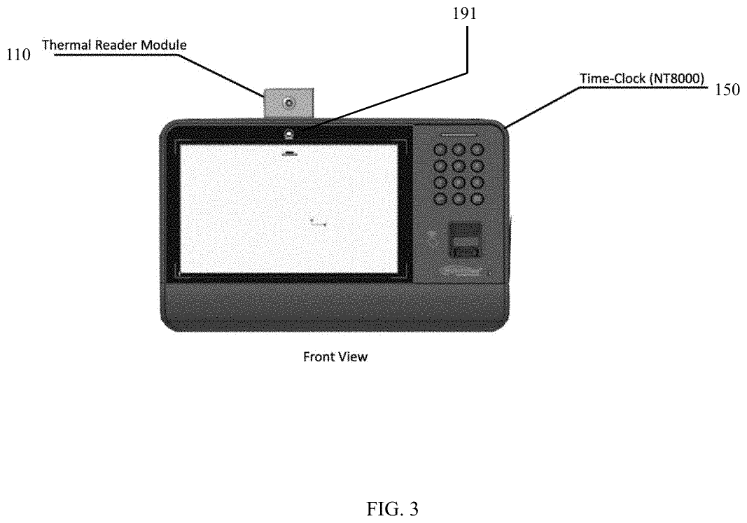

A functional block diagram of a first embodiment of the TRTC system is illustrated in FIG. 1. The TRTC system 100 is composed of three major elements: (1) a thermal reader device 110 capable of accurately measuring temperature of a human body from a distance of one foot or more and transmitting data collected to a host device; (2) a time-clock device 150 (Ascentis NT8000 or similar) capable of recording still images and full motion video, accept external peripheral devices, accept user input via touch screen, key pad, or voice command, establishing data connections with external devices and servers via wired or wireless networks, perform physical access control functions, and providing output via integrated screen and audible interactions; (3) a cloud-based web-enabled system 180 (e.g., Ascentis TimeKeeper/NOVAtime) running on a remote server capable of accepting transmissions from the time-clock (e.g., Ascentis NT8000 or similar) device. In the preferred embodiment, the TRTC system is configured to transmit signals to an access control system 190 that grants or denies access to the employer facility. The TRTC system can, for example, transmit signals that instruct the access control system 190 to unlock and/or open a door for an employee that meets all conditions for entry, or deny access to an employee with an elevated body temperature reading. The TRTC system may also comprise a time-clock camera 191 that may be configured to capture still photographs, live images, and/or video. Although not intended to be limiting, in FIGS. 1 and 3, the camera is located in proximity to and below thermal reader 110.

The Thermal Reader

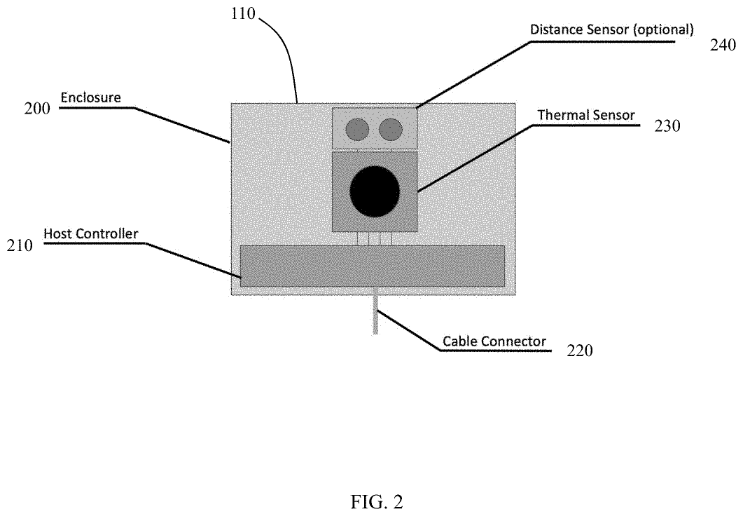

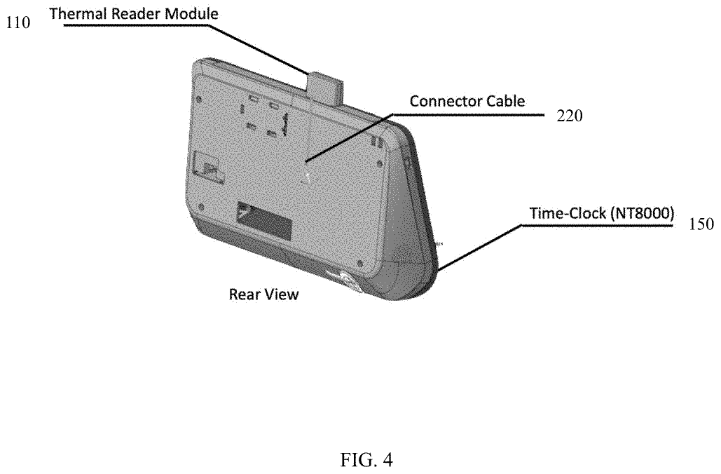

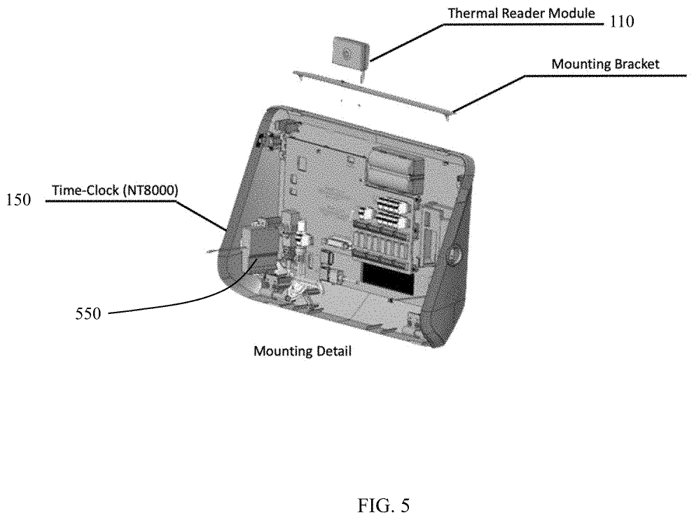

The thermal reader 110 device may be either integrated with the time-clock 150 or provided as a peripheral add-on configured to plug into the time-clock 150. The thermal reader 110 is a compact discrete circuit composed of a thermal sensor, a host controller, hard enclosure, an optional distance sensor, and cabling necessary to provide power, external control and data connectivity for the device. The thermal reader is controlled by the software on the time-clock devise 150 via a wired connection interface (USB, RS485, Wiegand, or similar) or alternatively a wireless RF data-transmission protocol (WiFi, Bluetooth, or similar). The same connection interface is used to transmit temperature data from the thermal reader 110 to the host time-clock device 150. A diagrammatic illustration of a thermal reader 110 is shown in FIG. 2, and an illustration of the thermal reader mounted to the time-clock is shown in FIGS. 3-5.

As illustrated in FIG. 2, the thermal reader comprises a housing or enclosure 200, a host controller 210 configured to communicate with the host time-clock, a cable connector 220 configured to plug into the time-clock 150, a thermal sensor 230, and a distance sensor 240 to determine how far away a person is standing when their temperature is being read.

The Time-Clock

Referring to FIGS. 3-5, the time-clock 150 has on-board a processor 550 and software capable of recognizing the presence of the thermal reader 110 and activating necessary functions to operate the device and acquiring rules for its interaction with customer's employees. The time-clock 150 may use images captured from the on-board camera 191 together with the thermal data captured from the thermal reader 110 to ensure proper employee positioning prior to taking the body temperature reading. The TRTC system 100 may inform the employee of correct or incorrect positioning via a combination of audio and visual signals.

The embedded software operating on the time-clock 150 interacts with the thermal reader 110 and provides feedback to the user/employee via visual, audio or stored data as to the status of the user's temperature. The specific condition of status is configurable by the customer in a web-enabled system and propagated to the time-clock for execution.

The Cloud-Based Web-Enabled System

Operating parameters and configuration options for the use of the thermal reader and reporting functions are managed by the remote cloud-based web-enabled system 180 (e.g., Ascentis TimeKeeper/NOVAtime). As part of the configuration, set-up, and activation, the customer/employer may be required to:

(1) confirm activation of the thermal reader feature,

(2) accept additional terms and conditions associated with the use of the temperature reader module,

(3) specify temperature threshold values across all employees globally, or to a subset of employees based on employee location, job group or class, employee position, etc.,

(4) specify when the temperature reading should be taken, for example, at time of clock-in clock-out, and any applicable action,

(5) action to be taken if an employee temperature reading exceeds the predefined threshold, possible actions including generating an audible alert, displaying an on-screen message, and/or transmitting an alert to a supervisor, etc.,

(6) action to be taken if reading fails threshold, actions including generating an audible alert, displaying an on-screen message, and/or transmitting an alert to a supervisor,

(7) action, if any, to be taken if employee does not grant a consent to having their temperature read by the thermal reader,

(8) set criteria for identifying the employee population—e.g., based on location, job group, position—for which the feature will be activated, and

(9) specify if the temperature reading function should be used with access control function and associated criteria.

Configuration and Initialization

For successful operation all elements of the system 100 must be properly configured and initialized. The thermal reader module 110 must be installed, recognized, and initialized by the time-clock 150. The time-clock 150 can also receive operational parameters from the cloud-based web-enabled system 180. If misconfiguration, functional fault, or other issues are detected then thermal reader module and related functions will default to the disabled state. Configuration state and status of each thermal reader time-clock system in an employer facility is transmitted to and captured by the cloud-based web-enabled system and made available for future reporting.

Time-Clock Initializing and Configuring

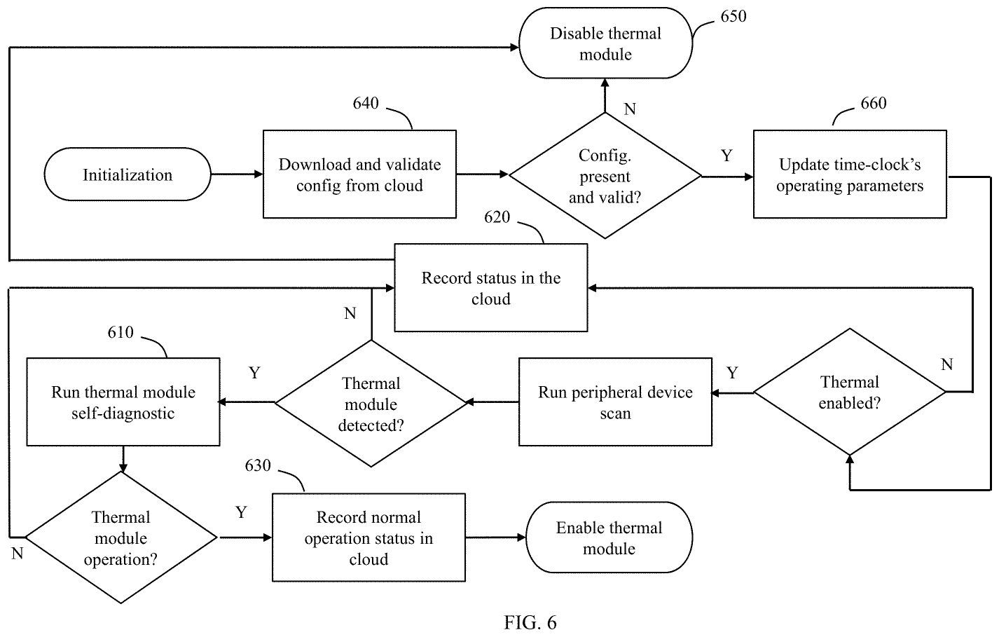

The process of initializing and configuring the time-clock when the peripheral thermal reader is attached to the time-clock is shown in FIG. 6 and described below.

(1) Upon installation of a peripheral thermal reader module, the time-clock may automatically detect the device. Alternatively, a manual initialization via the time-clock on-board software or the cloud-based web-enable system may be performed.

(2) The time-clock may perform an internal health-check to ensure the thermal reader module 110 is functioning as designed. A health-check 610 may include one or more of the following procedures: If an issue is detected, the time-clock may inform 620 the operator of the problem via a system prompt, visual, and/or audible alert. If no issues are detected the time-clock may report 630 the “Pass” or “Success” status to the operator or simply proceed to normal operating cycle. In either case, the time-clock initiates a data connection with the cloud-based web-enabled system to send data reflecting the current status of the clock.

(3) The time-clock 150 initiates a connection with the cloud-based web-enabled system to acquire 640 configuration and operational parameters for the thermal reader module.

If the configuration is not present, the time-clock may issue a warning or an informative prompt to the operator asking to supply the missing information and advising that the feature will be disabled 650 until the necessary parameters are provided.

If the configuration is present, the time-clock is configured to update 660 its operating setup to reflect the new parameters and may inform the operator that system is initialized and is ready for use.

Cloud-Based Web-Enabled System Configuration

The customer can access the cloud-based web-enabled system and access the configuration section of the system to provide configuration and operational parameters for the thermal reader including:

(1) Current state of the feature (enabled/disabled);

(2) Acceptance of additional terms and conditions;

(3) Temperature threshold (in degrees Celsius or Fahrenheit);

(4) Action, if any, to be taken if a reading exceeds established threshold temperature;

(5) Action, if any, to be taken if a reading fails;

(6) Action, if any, to be taken if employee does not grant a consent;

(7) Event(s) when the temperature reading should be taken (clock-in/clock-out, meal break , etc);

(8) Current state of association between access control and temperature reading functions; and

(9) Criteria for identifying the employee population for whom the feature is active

Employee Consent

Prior to taking the first-time measure of the body temperature of each individual employee, the time-clock is configured to solicit consent from that employee. The feature is not active for employees that have not granted explicit consent. The employer/customer may use the cloud-based web-enabled system to instruct time-clocks to attempt to re-acquire consent regardless of its current status.

Acquiring Consent Using Time-Clock

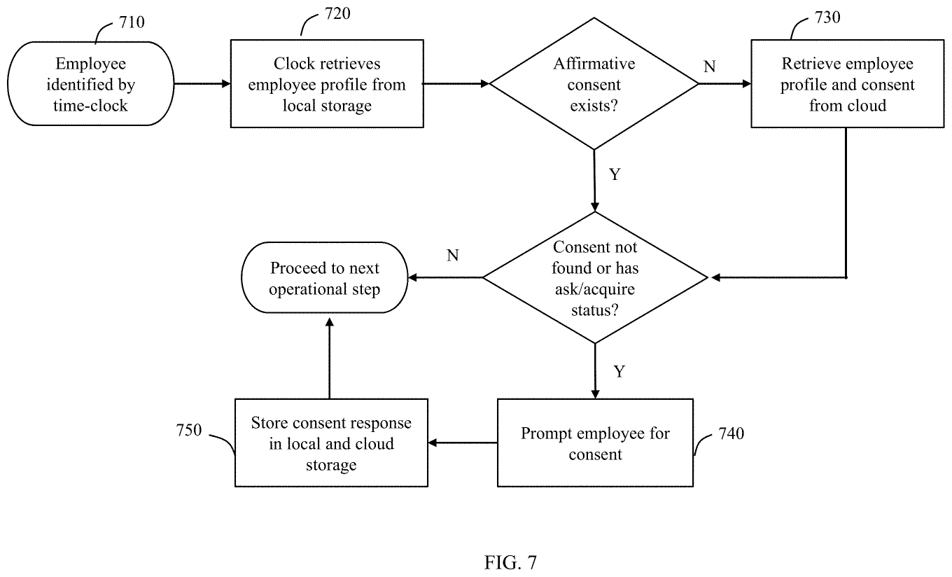

The procedure for acquiring an employee's consent using a thermal read time-clock system 100 is illustrated in FIG. 7 and described below. As a part of a standard task, employee provides clock with profile identifying information and the employee identified 710. The time-clock checks its local data to verify 720 whether or not the employee has consented to submit to a temperature reading. If the consent information is not present in local storage, the time-clock initiates a connection to the cloud-based web enabled system and issue a request 730 to query for the consent information. If the consent information is not found on the cloud-based system, or consent data is found but reflects “ask/acquire” status, the TRTC system is configured to alert the employee with visual and/or audio queue and present the consent information on the screen together with a request 740 for the employee to either agree or decline. The employee can respond to the consent request via available touchscreen, keypad, or voice command in order to proceed with their task. The response from the employee is recorded 750 in time-clock's local storage and a connection to the cloud-based web-enabled system is initiated to record the response in the remote server database.

Collection Using Cloud-Based Web-Enabled User Interface

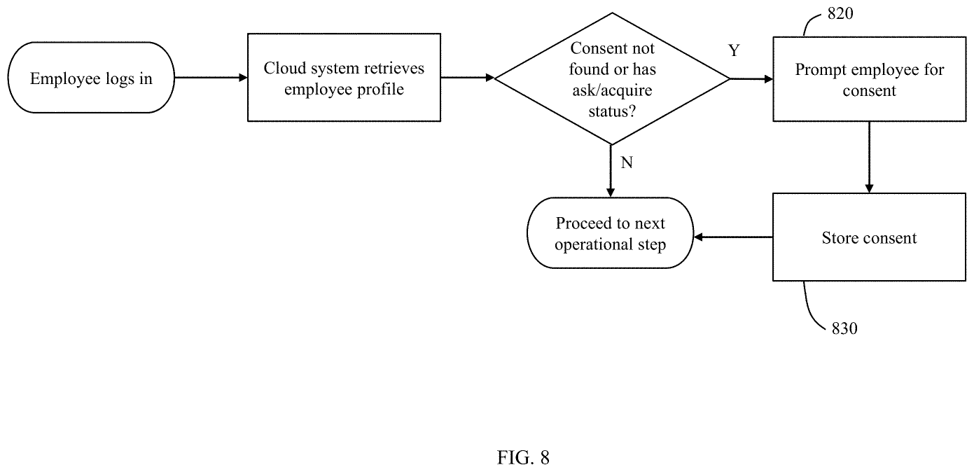

An employee may also provide consent to a temperature reading via a web-enabled user interface. In this embodiment, the TRTC system 100 prompts 820 the employee with consent information requiring an agree/decline response. The employee must respond to the consent request in order to proceed with their task. The response from the employee is recorded 830 in the system's database. The process of acquiring consent via the web interface is illustrated in FIG. 8.

Reporting Temperature Readings

The employer/customer may access the cloud-based web-enabled system to produce real-time, historical, detailed, or aggregated reports containing employee information and their consent status, which is typically: agreed, declined, ask/acquire or similar.

Management

The employer/customer may access the cloud-based web-enabled system to manage consent status of individual employee or group of employees. The TRTC system enables the employer to search for individual employees based on various criteria including location, job function, management structure, consent status, etc. When results of the query are presented, the employer may be given an option to re-acquire consent of selected employees or the entire group identified by the search criteria or other parameters. If the employer selects the “re-acquire consent” option, then the cloud-based web-enabled system archives the previous consent status for employee(s) affected and record the new status as “ask/acquire”).

Body Temperature

The body temperature reading is acquired at the time of employee's interaction with the thermal reader time-clock 100 (clock-in, clock-out, meal-break, transfer, etc). Even though the thermal reader time-clock 100 may take the temperature reading during each employee interaction, the device may only trigger the configured action during the configured event(s) which may include: on-screen visual or audible warning, alert to a supervisor(s), denying physical access, or other. In order to ensure an accurate reading, the time-clock may use the thermal reader module together with its camera 191 to capture and analyze still or real-time images for purposes of position and or face detection. The thermal reader time-clock may also capture an image of the employee being subjected to the temperature reading. The time-clock may capture the events when employee's reading exceeds established threshold as well as actual reading data. This captured data may be transmitted to the cloud-based web-enabled system and made available for subsequent reporting and analysis.

Identifying an Employee Time-Clock Event

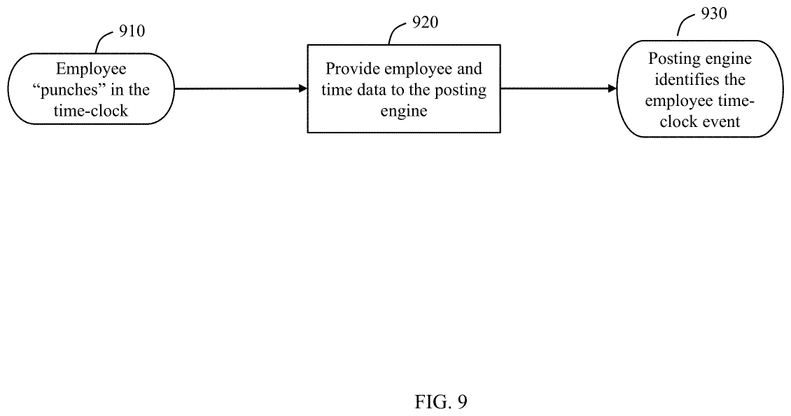

As a part of a standard task, employees provide 910 identifying information to the time-clock. The time-clock uses the employee and time data to consult 920 its posting engine (either on-board or hosted in the remote cloud-based web-enabled system) to determine 930 the action/task being carried by the employee and its associated event (clock-in, clock-out, meal break, transfer, etc). The process of providing identifying information is illustrated in FIG. 9.

Ensuring Accurate Temperature Reading

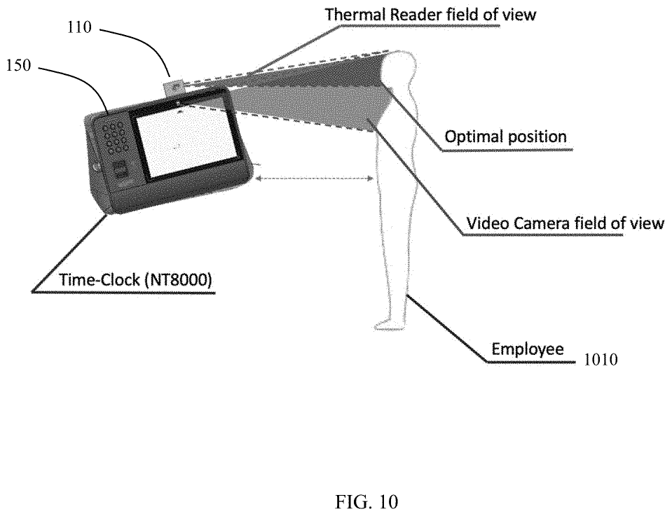

The time-clock 150 is configured to utilize its on-board image capture capabilities together with the data from the thermal reader 110 to determine location and position of the employee 1010 prior to taking the temperature reading. The proper location of the employee 1010 relative to the TRTC system is shown in FIG. 10.

The time-clock 150 is configured to use one of the two following algorithms to determine optimal employee position. One algorithm is for a time-clock with a distance sensor 240, and the second if for a time-clock without distance sensor.

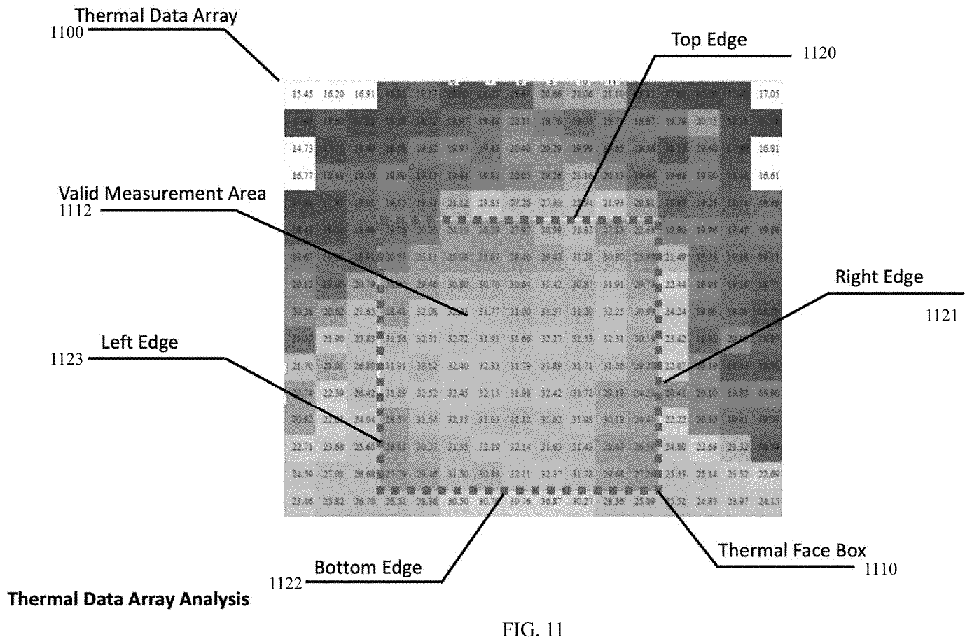

For a time-clock with distance sensor 240: The time-clock 150 acquires an image of the employee 1010 from the on-board video camera 191 and a facial detection algorithm used to ensure that the employee's face is within the field of view of the camera 191. Data 1100 from the thermal reader 110 is then analyzed to generate a “thermal face box” 1110. The boundaries of the thermal face box include four edges 1120-1123 around employee's face 1112. These boundaries 1120-1123 are generated by dividing the image into sub-regions called cells and then comparing the temperature readings of neighboring cells to identify significant differences in values. A grid of cells, the corresponding temperature readings, and thermal face box 1110 is shown in FIG. 11.

The size and completeness of this “thermal face box” 1110 is used to confirm correct positioning. If an employee 1010 is standing too close to the time-clock 150, the timeclock is configured to instruct the employee to move backwards until the video camera 191 is able to detect a face and generate the “thermal face box” 1110. If an employee 1010 is standing too far to the left or right of the time-clock 150, the time-clock is configured to instruct the employee to move one direction of the other until it can detect a face and generate the “thermal face box”. If the employee 1010 is standing too far from the time-clock, the time-clock is configured to instruct the employee to move closer to the video camera 191.

For time-clock with distance sensor: The distance data may be used—in addition to the algorithm described above—to verify that the employee is positioned at optimal distance from the time-clock, or guide the employee to the optimal distance if necessary. In scenarios where the employee may not be able to achieve optimal distance from the clock due to physical restriction of the space or other factors, the distance data may be used together with the temperature array data to ensure accuracy of the temperature reading and allow measurements to be taken in those conditions.

If the position of the employee is determined to be sub-optimal for an accurate reading, the time-clock 150 is configured to prompt the employee with on-screen or audible instructions directing employee to a better position. The directions may include real-time images of the employee's current position and queues describing the desired position.

Body Temperature Reading

The thermal reader time-clock 100 determines if an employee clock-in event, for example, qualifies for a temperature reading. Whether or not the event qualifies is based on employee criteria and event criteria specified in the operational parameters. If necessary, the time-clock establishes a connection with cloud-based web-enabled system to download configuration values and operational parameters related to the temperature reader module.

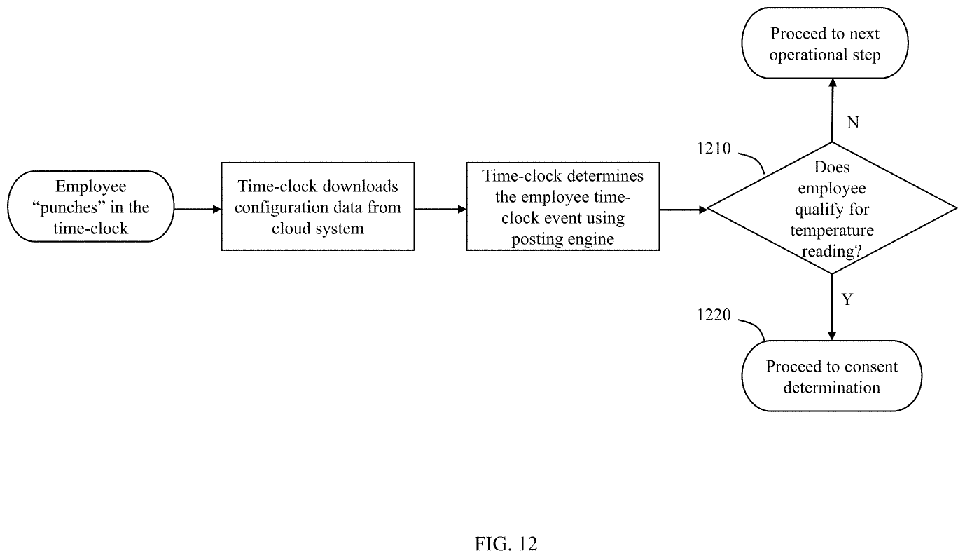

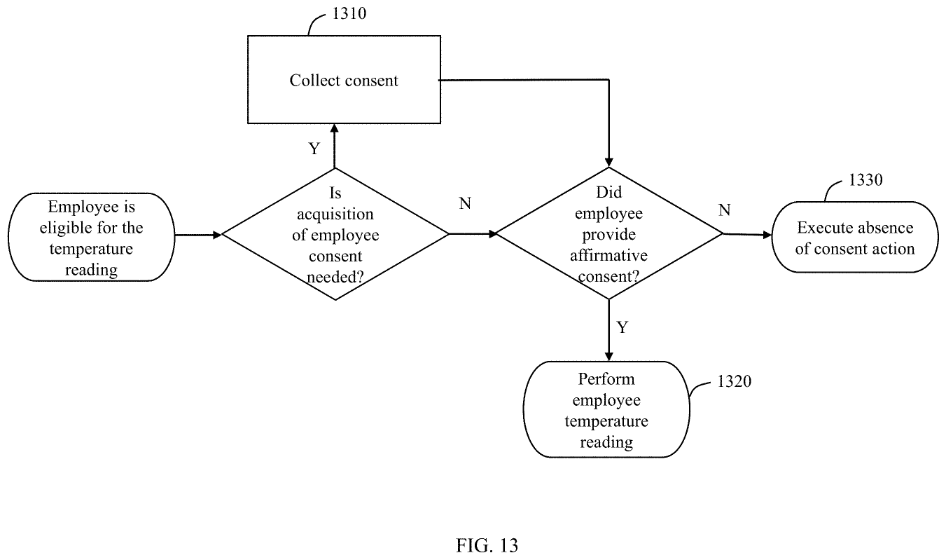

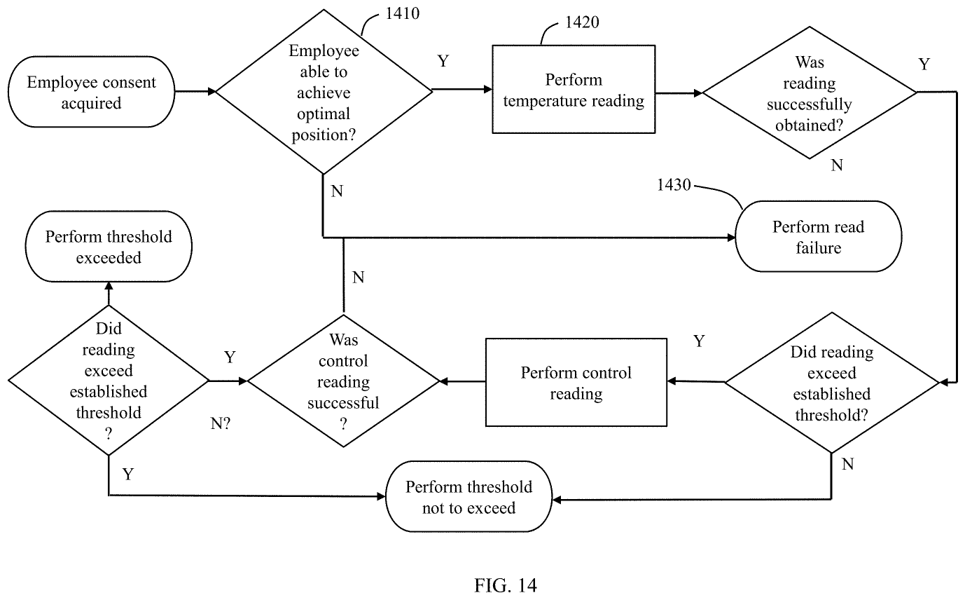

The time-clock 150 is configured to determine 1210 if an active and valid consent record exists. This procedure is illustrated in FIG. 12. If the time-clock determines that consent must be collected, then a routine to acquire 1220 the necessary consent is activated. This procedure is illustrated in FIG. 13. If consent is not received, the time-clock executes 1330 an action specified in pre-defined operational parameters. If consent is received 1310, the time-clock ensures that the employee is in the optimal position using the mechanism described above. If and when 1410 an optimal employee position is achieved, then a “capture reading” command is sent 1420 to the thermal reader and data is received 1320 by the time-clock. See FIG. 14. If the optimal position is not achieved, then a “reading failed” status is captured 1430 by the time-clock.

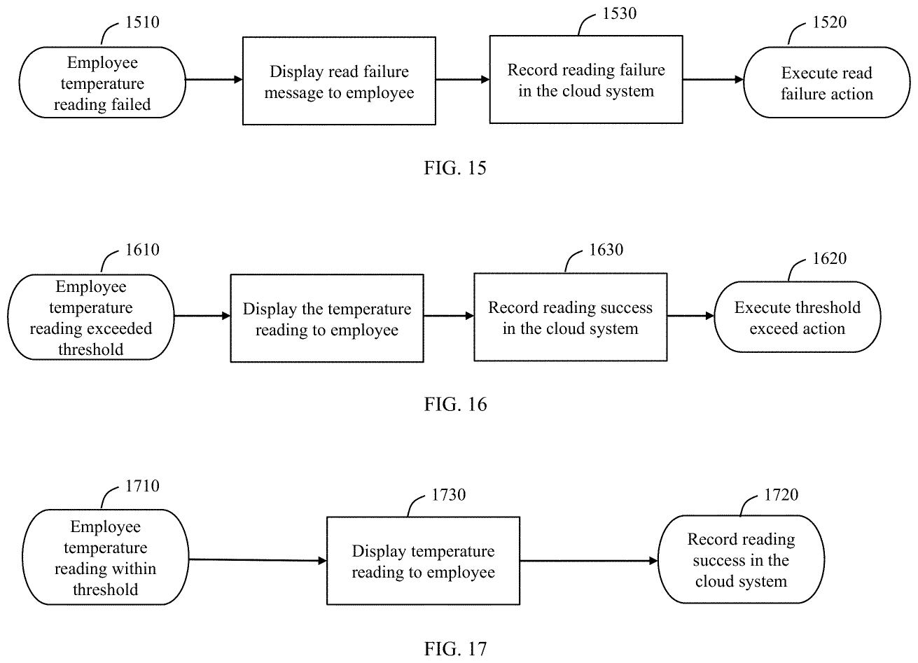

If a temperature reading results in “reading failed” 1510, then the time-clock executes 1520 the action specified by the configuration and operational parameters. See FIG. 15. If, however, the temperature data was successfully acquired by the time-clock, it processes it as necessary to determine the actual numerical value in degrees C. and/or F. and compares to threshold specified in the configuration and operational parameters.

If the threshold is exceeded 1610, then the time-clock takes 1620 another control reading from the employee. See FIG. 16. If the control reading also exceeds the established threshold then an action specified in the configuration and operational parameters is executed.

If the control reading does not exceed the established temperature threshold defined for that employee, then another “tie-breaker” temperature reading is taken. See FIG. 17. If the “tie-breaker” temperature reading exceeds 1710 the threshold, then an action(s) specified in the configuration and operational parameters is executed 1720.

The time-clock shows 1730 the prompt to the employee indicating that temperature reading was successfully taken and displays the measured temperature value to on screen.

The time-clock may store 1530, 1630, 1730 the success/failure status of the reading, the actual temperature value, and the threshold test outcome in local storage and transmit it to the cloud-based web-enabled system to be recorded and stored for future reporting and auditing.

Reporting

An employer/customer may access the cloud-based web-enabled system to produce real-time, historical, detailed, or aggregated reports that contain employee information and their body temperature reading data, the data including a Threshold field having a value of pass/fail, and a Temperature reading status having a value of success/fail.

Employer Interface

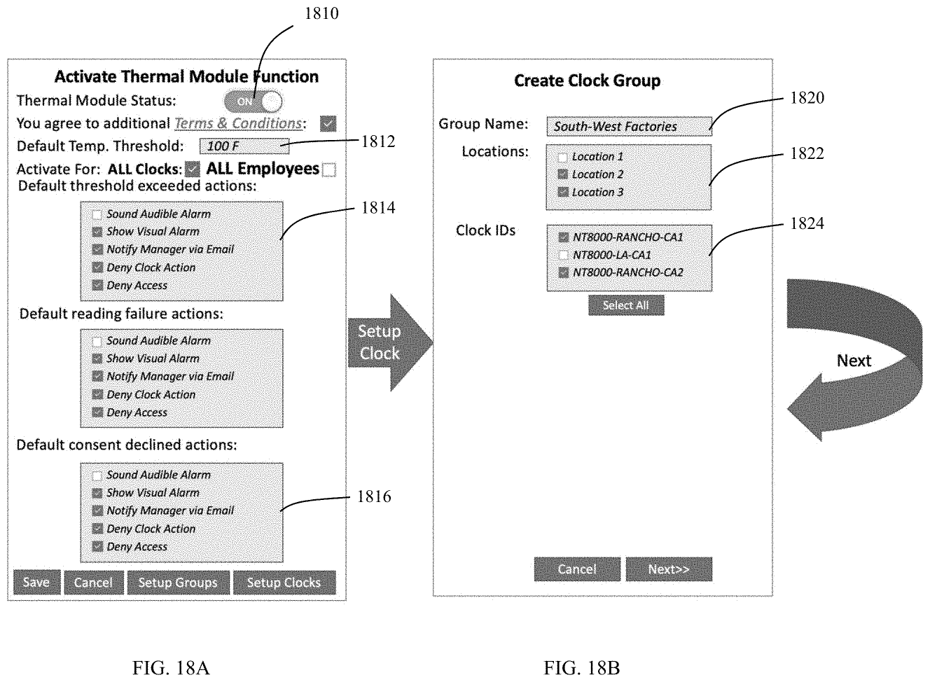

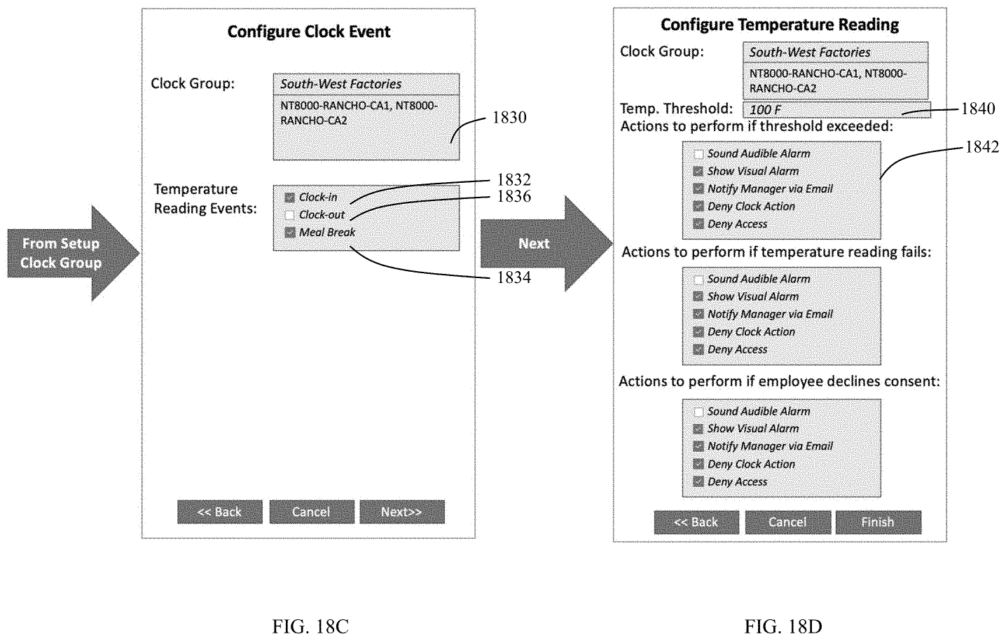

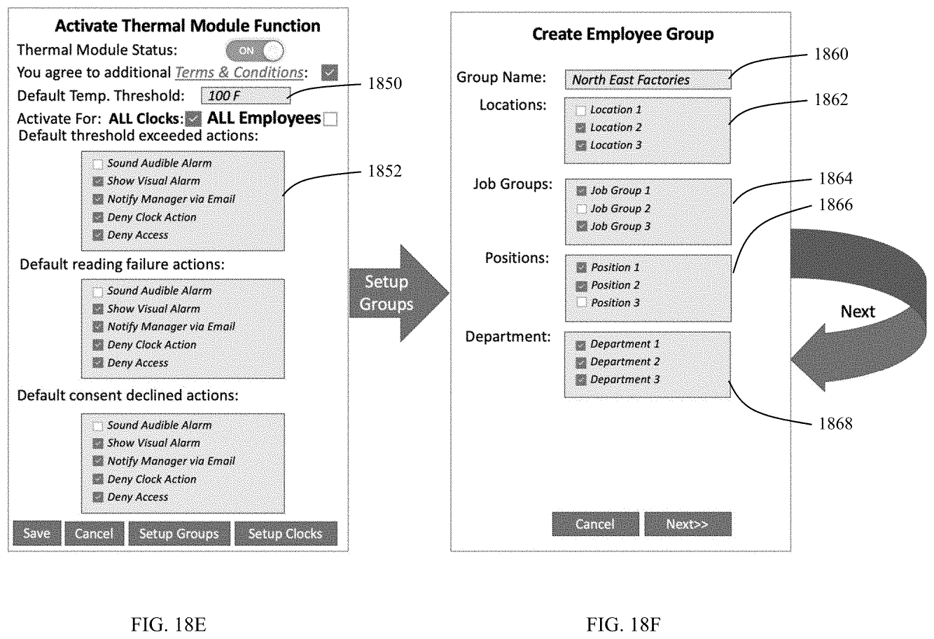

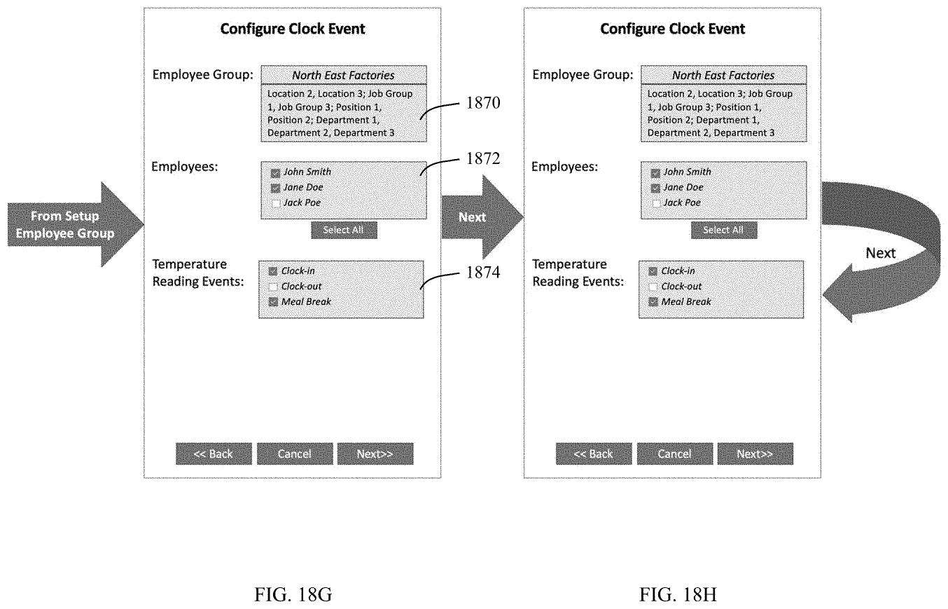

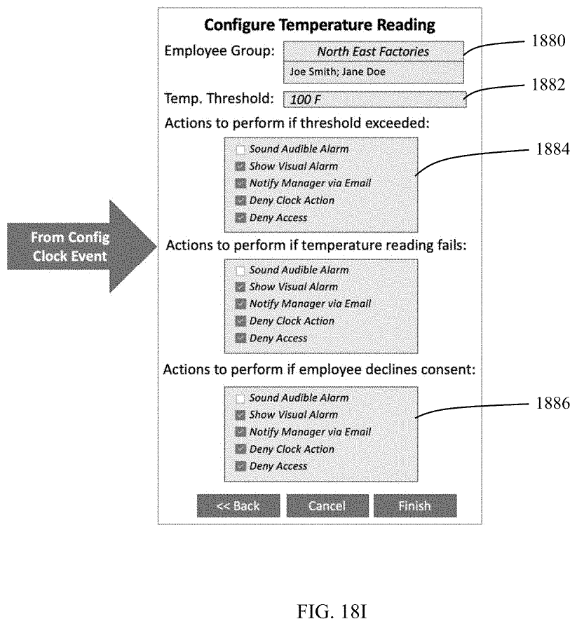

Illustrated in FIGS. 18A-18I is a sequence of dialogue boxes present in user interface enabling an employer to define various actions to be taken based, in part, on an employee's body temperature and the criteria that trigger those actions. Referring to FIG. 18A, the employer can enable the thermal reader 1810, define a temperature threshold value 1812, and actions to be taken if an employee body temperature reading exceeds the threshold 1814, as well as actions to be taken if an employee does not consent to a thermal scan 1816.

Referring to FIG. 18B, the employer can define a class of employees 1820 referred to herein as a “group” by location 1822 or badge number 1824, for example, to which the temperature threshold value 1812 and associated actions 1814, 1816 apply.

Referring to FIG. 18C, the employer can define when employee temperature readings are to be acquired 1830. As shown, temperature readings are to be acquired at time of clock-in 1832 and meal breaks 1834, but not clock-out 1836, for the class of employees identified as “South-West Factories”.

Referring to FIG. 18D, the employer can define what actions are to be taken if the employee temperature reading is above the 100 degree threshold 1840. For example, the TRTC system 100 is configured 1842 here to show a visual alarm to the employee, notify the employee's manager by email, prevent or otherwise deny the employee from clocking-in to work, and deny access to the employee to prevent entry into the employer facility.

Illustrated in FIG. 18E is a user interface by which an employer may define various actions 1852 to be taken when an employee body temperature reading exceeds the predefined threshold 1850.

Illustrated in FIG. 18F is a user interface by which an employer may define classes, i.e., groups 1860, of employees based on employee location 1862, job group 1864, position 1866, and department 1868, for example. Once a class is defined, a threshold 1850 body temperature and various actions may be defined and customized for that class of employees.

Illustrated in FIGS. 18G-18H is a user interface by which an employer may configure clock events for individual employees as members of a class of employees. For example, the employees “John Smith” and “Jane Doe” 1872—which are members of the North East Factories group 1870—are required to provide a temperature reading at time of clock-in and meal break 1874, while the employee “Jack Poe” is exempt from providing the temperature reading even though he is also a member of the employee class referred to herein as the “North East Factories”. The applicable actions 1884 to be taken in response to the body temperature reading for “John Smith” and “Jane Doe” exceed the threshold 1882 are shown in FIG. 18I. If the employee's temperature reading exceeds the threshold 1882, the resulting actions 1884 are taken. If an employee fails to provide consent to a temperature reading, actions 1886 are taken.

The system can take the form of an entirely hardware embodiment, an entirely software embodiment or an embodiment containing both hardware and software elements. In one embodiment, the system is implemented in software, which includes but is not limited to firmware, resident software, microcode, etc.

Furthermore, the system can take the form of a computer program product accessible from a computer-usable or computer-readable medium providing program code for use by or in connection with a computer or any instruction execution system. For the purposes of this description, a computer-usable or computer readable medium can be any apparatus that can contain, store, communicate, propagate, or transport the program for use by or in connection with the instruction execution system, apparatus, or device.

The medium can be an electronic, magnetic, optical, electromagnetic, infrared, or semiconductor system (or apparatus or device) or a propagation medium. Examples of a computer-readable medium comprise a semiconductor or solid-state memory, magnetic tape, a removable computer diskette, a random access memory (RAM), a read-only memory (ROM), a rigid magnetic disk and an optical disk. Current examples of optical disks comprise compact disk-read only memory (CD-ROM), compact disk-read/write (CD-R/W) and DVD.

A data processing system suitable for storing and/or executing program code comprises at least one processor coupled directly or indirectly to memory elements through a system bus. The memory elements can include local memory employed during actual execution of the program code, bulk storage, and cache memories that provide temporary storage of at least some program code in order to reduce the number of times code is retrieved from bulk storage during execution.

Input/output or I/O devices (including but not limited to keyboards, displays, pointing devices, etc.) can be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

Described above, aspects of the present application are embodied in a World Wide Web (“WWW”) or (“Web”) site accessible via the Internet. As is well known to those skilled in the art, the term “Internet” refers to the collection of networks and routers that use the Transmission Control Protocol/Internet Protocol (“TCP/IP”) to communicate with one another. The internet 20 can include a plurality of local area networks (“LANs”) and a wide area network (“WAN”) that are interconnected by routers. The routers are special purpose computers used to interface one LAN or WAN to another. Communication links within the LANs may be wireless, twisted wire pair, coaxial cable, or optical fiber, while communication links between networks may utilize 56 Kbps analog telephone lines, 1 Mbps digital T-1 lines, 45 Mbps T-3 lines or other communications links known to those skilled in the art.

Furthermore, computers and other related electronic devices can be remotely connected to either the LANs or the WAN via a digital communications device, modem and temporary telephone, or a wireless link. It will be appreciated that the internet comprises a vast number of such interconnected networks, computers, and routers.

The Internet has recently seen explosive growth by virtue of its ability to link computers located throughout the world. As the Internet has grown, so has the WWW. As is appreciated by those skilled in the art, the WWW is a vast collection of interconnected or “hypertext” documents written in HTML, or other markup languages, that are electronically stored at or dynamically generated by “WWW sites” or “Web sites” throughout the Internet. Additionally, client-side software programs that communicate over the Web using the TCP/IP protocol are part of the WWW, such as JAVA.RTM. applets, instant messaging, e-mail, browser plug-ins, Macromedia Flash, chat and others. Other interactive hypertext environments may include proprietary environments such as those provided in America Online or other online service providers, as well as the “wireless Web” provided by various wireless networking providers, especially those in the cellular phone industry. It will be appreciated that the present application could apply in any such interactive communication environments, however, for purposes of discussion, the Web is used as an exemplary interactive hypertext environment with regard to the present application.

A web site is a server/computer connected to the Internet that has massive storage capabilities for storing hypertext documents and that runs administrative software for handling requests for those stored hypertext documents as well as dynamically generating hypertext documents. Embedded within a hypertext document are a number of hyperlinks, i.e., highlighted portions of text which link the document to another hypertext document possibly stored at a website elsewhere on the Internet. Each hyperlink is assigned a URL that provides the name of the linked document on a server connected to the Internet. Thus, whenever a hypertext document is retrieved from any web server, the document is considered retrieved from the World Wide Web. Known to those skilled in the art, a web server may also include facilities for storing and transmitting application programs, such as application programs written in the JAVA.RTM. programming language from Sun Microsystems, for execution on a remote computer. Likewise, a web server may also include facilities for executing scripts and other application programs on the web server itself.

A remote access user may retrieve hypertext documents from the World Wide Web via a web browser program. A web browser, such as Netscape's NAVIGATOR.RTM. or Microsoft's Internet Explorer, is a software application program for providing a user interface to the WWW. Upon request from the remote access user via the web browser, the web browser requests the desired hypertext document from the appropriate web server using the URL for the document and the hypertext transport protocol (“HTTP”). HTTP is a higher-level protocol than TCP/IP and is designed specifically for the requirements of the WWW. HTTP runs on top of TCP/IP to transfer hypertext documents and user-supplied form data between server and client computers. The WWW browser may also retrieve programs from the web server, such as JAVA applets, for execution on the client computer. Finally, the WWW browser may include optional software components, called plug-ins, that run specialized functionality within the browser.

The foregoing description of the preferred embodiment of the invention has been presented for the purposes of illustration and description. It is not intended to be exhaustive or to limit the invention to the precise form disclosed. Many modifications and variations are possible in light of the above teaching. It is intended that the scope of the invention not be limited by this detailed description, but by the claims and the equivalents to the claims appended hereto.

1. A system comprising:
a time-clock comprising a display, wherein the time-clock is configured to:
i. identify a user;
ii. record a clock-in time associated with the user after the system identifies the user; and
iii. record a clock-out time associated with the user after the system identifies the user;
a thermal reader configured to acquire at least one body temperature reading of the user;
a housing comprising the thermal reader and time clock; and
a database comprising:
a. a predefined temperature threshold;
b. at least one instruction specifying an action to be taken if and when the body temperature reading of the user exceeds the predefined temperature threshold;
c. a plurality of user groups comprising a first user group and a second user group;
wherein the system is configured to:
1. acquire a body temperature reading of a user if the user is identified by the time-clock as being a member of the first user group; and
2. exempt a user from a body temperature reading if the user is identified by the time-clock as being a member of the second user group.

2. The system in claim 1, wherein the time-clock comprises a badge reader configured to identify the user based on a badge associated with the user.

3. The system in claim 2, wherein the time-clock is configured to record the clock-in time and the clock-out time if and when the predefined temperature threshold exceeds the at least one body temperature reading of the user.

4. The system in claim 3, wherein the time-clock is configured to execute one or more actions if and when the at least one body temperature reading of the user exceeds the predefined temperature threshold.

5. The system in claim 4, wherein the one or more actions comprise:
displaying an on-screen message to the user indicating an elevated body temperature.

6. The system in claim 5, wherein the one or more actions comprise:
transmitting an alert to a supervisor of the user.

7. The system in claim 6, wherein the one or more actions comprise:
preventing the time-clock from recording a clock-in event associated with the user.

8. The system in claim 7, wherein the one or more actions comprise:
denying the user access to an employer facility.

9. The system in claim 1, wherein the predefined temperature threshold corresponds to a fever.

10. The system in claim 9, wherein the predefined temperature threshold is equal to about 100 degrees Fahrenheit.

11. The system in claim 1, wherein the time-clock is configured to prompt the user for consent before taking a body temperature reading of the user.

12. A system comprising:
a time-clock comprising a display, wherein the time-clock is configured to:
i. identify a user based on a badge associated with the user;
ii. record a clock-in time and clock-out time associated with the user after the system identifies the user;
iii. prompt the user for consent before acquiring a body temperature reading after the system identifies the user;
iv. detect a position of the user relative to the time-clock; and
v. instruct the user to move based on the detected position of the user if the position of the user relative to the time-clock will not permit the acquisition of a thermal image of the user;
a thermal reader configured to:
a. acquire a thermal image of the user if the user has consented to the body temperature reading;
b. determine a portion of the thermal image associated with a face of the user in order to facilitate acquisition of the body temperature reading of the user;
c. acquire the body temperature reading of the face of the user;
a housing comprising the thermal reader and time clock; and
a database comprising a predefined temperature threshold;
wherein the system is configured to record a plurality of user groups, each user group characterized by one or more attributes; wherein the plurality of user groups comprises a first user group and a second user group; wherein the system is further configured to acquire the body temperature reading of a user if the user is identified by the time-clock as being a member of the first user group;
wherein the time-clock is configured to record the clock-in time associated with the user if and when the predefined temperature threshold exceeds the body temperature reading of the user;
wherein the time-clock is configured to execute a plurality of actions if and when the body temperature reading of the user exceeds the predefined temperature threshold, said actions comprising:
1. displaying a message to the user indicating an elevated body temperature;
2. transmitting an alert to a supervisor of the user;
3. preventing the time-clock from recording a clock-in event associated with the user; and
4. denying the user access to an employer facility.

13. The system of claim 1, wherein the time-clock is configured to execute one or more actions if and when the at least one body temperature reading of the user exceeds the predefined temperature threshold.

14. The system of claim 13, wherein the one or more actions comprise:
displaying an on-screen message to the user indicating an elevated body temperature.

15. The system in claim 13, wherein the one or more actions comprise:
transmitting an alert to a supervisor of the user.

16. The system in claim 13, wherein the one or more actions comprise:
preventing the time-clock from recording a clock-in event associated with the user.

17. The system in claim 13, wherein the one or more actions comprise:
denying the user access to an employer facility.

18. The system of claim 12, further configured to exempt a user from a body temperature reading if the user is identified by the time-clock as being a member of the second user group.

19. The system of claim 18, further configured to acquire the body temperature reading of a user at a time of clock-in and meal break if the user is identified by the time-clock as being a member of the first user group.

20. The system of claim 19, wherein the one or more attributes is based on a geographic location at which the user is employed.
